(19) United States
(12) Patent Application Publication
(10) Pub. No.: US 2017/0087108 A1
(43) Pub. Date: Mar. 30, 2017

(54) METHODS OF IMPROVING PHYSIOLOGICAL CONDITIONS RELATED TO PREGNANCY THROUGH DIETARY SUPPLEMENTATION

(71) Applicant: Natural Alternatives International, Inc., San Marcos, CA (US)

(72) Inventor: Roger Harris, Newmarket (GB)

(21) Appl. No.: 15/266,861

(22) Filed: Sep. 15, 2016

Related U.S. Application Data
(60) Provisional application No. 62/233,071, filed on Sep. 25, 2015.

(51) Int. Cl. A61K 31/197; A61K 47/26; A23L 33/00; A23K 50/75; A23L 33/17; A23K 20/142; A23K 50/30; A61K 45/06; A23L 33/175

(52) U.S. Cl. CPC A61K 31/197; A61K 45/06; A61K 47/26; A23L 33/40; A23L 33/175; A23L 33/17; A23K 20/142; A23K 50/30; A23K 50/75; A23V 2002/00

(57) ABSTRACT

The present invention provides processes where free amino acid beta-alanine, or a salt or ester thereof, is administered to an individual as a dietary supplement over a period of time in an amount effective to improve physiological conditions related to pregnancy and/or improve physiological conditions of offspring of the individual.

### CROSS-REFERENCE TO RELATED APPLICATIONS

This application claims benefit of U.S. Provisional Patent Application No. 62/233,071, filed Sep. 25, 2015, which is herein incorporated by reference.

### FIELD OF THE INVENTION

The present invention relates to dietary supplementation and physiology, and, more specifically, to methods of improving physiological conditions related to pregnancy and development of offspring.

### BACKGROUND OF THE INVENTION

There are many conditions related to pregnancy and development of offspring, both prior to birth and following birth, that may affect the health and viability of a fetus and/or offspring. Physiological conditions may exist and/or manifest themselves during pregnancy and/or after birth that may affect the fetus and/or offspring during pregnancy and/or after birth. Similarly, certain stresses occur in oviparous animals during the egg production and incubation stage.

For human and non-human individuals, it is well-known that morbidity and mortality are higher for babies born pre-term. Infants surviving premature birth can be left with severe, life-long disabilities of the nervous system, occurring not only as a result of the prematurity, but also from co-existing obstetric problems such as intrauterine infection, chronic fetal hypoxia, or with the problems that arise during resuscitation of an infant. In oviparous animals, stresses in egg laying, incubation and hatching can lead to higher levels of morbidity.

For non-human individuals, such as livestock, there are many factors that are of critical importance to farmers. These include, increasing the size of a litter, reducing the number of non-live births, reducing infant mortality, increasing early survival rates, increasing hatchling viability, increasing intelligence, promoting early growth, increasing lean muscle, enhanced nutritive value of offspring, etc.

Investigations have shown that creatine supplementation during pregnancy may improve fetal and neonatal morbidity and reduce mortality in high-risk human pregnancy. (Dickinson et al., 2014).

Investigations examining β-alanine (herein also referred to as beta-alanine or BA) ingestion have been consistent in demonstrating significantly enhanced athletic performance during high intense activity (e.g., resistance exercise, repeated sprints) to a greater magnitude than a placebo (Hill et al., 2007; Hoffman et al., 2006; 2008a; 2008b; 2012; Kendrick et al., 2008; Stout et al., 2006; 2007). The efficacy of β-alanine ingestion appears centered on its ability to enhance the quality of a workout and sport performance by delaying skeletal muscle fatigue when supplemented in an effective amount over a sufficient period of time as with a dietary supplement. The ergogenic properties of β-alanine taken as a single dose by itself appear to be very limited, but when consumed in sufficient dosages over time, β-alanine combines in the skeletal muscle with L-histidine to form the dipeptide carnosine (beta-alanylhistidine) and appear to have ergogenic effects (Dunnett and Harris, 1999). The primary role of carnosine is in the maintenance of acid-base homeostasis through enhanced intra-muscular hydrogen ion (H⁺) buffering capacity (Harris et al., 2006). Increasing intra-muscular carnosine concentration through β-alanine supplementation has demonstrated ergogenic potential for maximal exercise lasting 60 sec-240 sec (Hobson et al., 2012). Because carnosine is located in other tissues in addition to skeletal muscle, such as the brain and heart, it may also have additional physiological roles.

The following art is incorporated by reference in its entirety, but especially for the information characterized by the title, which would be easily understood by one of skill in the art. Artioli G. G., Gualano B., Smith A., Stout J. R., Junior A. H. L., The Role of β-alanine Supplementation on Muscle Carnosine and Exercise Performance. Med Sci Sports Exerc 42(6):1162-1173, 2010; Baguet, A., et al. (2010). “Important role of muscle carnosine in rowing performance.” J of App. Physio. 109(4): 1096-1101; Boldyrev A. A., Stvolinsky S. L., Fedorova T. N., Suslina Z. A., (2010) Carnosine as a natural antioxidant and geroprotector: from molecular mechanisms to clinical trials. Rejuvenation Res. 13:156-158; Cassler N. M., Sams R., Cripe P. A., McGlynn A. F., Perry A. B., Banks B. A., Patterns and perceptions of supplement use by U.S. Marines deployed to Afghanistan. Mil Med. 2013 178(6):659-64; Derave, W., et al. (2010). “Muscle carnosine metabolism and β-alanine supplementation in relation to exercise and training.” Sports medicine 40(3): 247-263; Derave W., Ozdemir M. S., Harris R. C., Pottier A., Reyngoudt H., Koppo K., Wise J. A., Achten E., Beta-Alanine supplementation augments muscle carnosine content and attenuates fatigue during repeated isokinetic contraction bouts in trained sprinters. J Appl Physiol 103(5):1736-1743, 2007; Dunnett M., Harris R. C. (1999), Influence of oral beta-alanine and L-histidine supplementation on the carnosine content of the gluteus medius. Equine Vet J Suppl 30:499-504; Del Favoro S., Roschel H., Solis M., Hayashi A., Artiolo G., Otaduy M., Benatti F., Harris R., Wise J., Leite C., Pereira R., de Sa-Pinto A., Lancha-Junior A., Gualano B. (2012), Beta-alanine (Carnosyn™) supplementation in elderly subjects (60-80 years): effects on muscle carnosine content and physical capacity. Amino Acids 43:49-56; Estrada A., Kelley A. M., Webb C. M., Athy J. R., Crowley J. S., Modafinil as a replacement for dextroamphetamine for sustaining alertness in military helicopter pilots. Aviat Space Environ Med. 2012, 83(6):556-64; Evans R. K., Scoville C. R., Ito M. A., Mello R. P., Upper body fatiguing exercise and shooting performance. Mil. Med. 168:451-456, 2003; Gillingham R. L., Keefe A. A., Tikuisis P., Acute caffeine intake before and after fatiguing exercises improves target shooting engagement time. Aviat. Space Environ. Med. 75:865-871, 2004; Green S. B., Salkind N. J., Akey T. M.,: Using SPSS for Windows: Analyzing and Understanding Data (2^{nd} ed.), Upper Saddle River, N.J.: Prentice Hall, 2000; Harman, E. A., et al. (2008). “Effects of two different eight-week training programs on military physical performance.” The Journal of Strength & Conditioning Research 22(2): 524-534; Harris R. C., Tallon M. J., Dunnett M., Boobis L., Coakley J., Kim H. J., Fallowfield J. L., Hill C. A., Sale C., Wise J. A., (2006), The absorption of orally supplied β-alanine and its effect on muscle carnosine synthesis in human vastus lateralis. Amino Acids 30(3)279-289; Hayman M., Two minute clinical test for measurement of intellectual impairment in psychiatric disorders. Arch Neuro Psychiatry 1942;47:454-64; Hill C. A., Harris R. C., Kim H. J., Harris B. D., Sale C., Boobis L. H., Kim C. K., Wise J. A., (2007), Influence of β-alanine supplementation on skeletal muscle carnosine concentrations and high intensity cycling capacity. Amino Acids 32(2):225-233; Hobson R. M., Saunders B., Ball G., Harris R. C., Sale C., (2012), Effects of Beta-alanine supplementation on exercise performance: a meta-analysis. Amino Acids. 43(1):25-37; Hoffman J. R., Emerson N. S., Stout J. R., β-alanine supplementation. Curr. Sports Med. Rep. 11:189-195, 2012; Hoffman J. R., Ratamess N. A., Faigenbaum A. D., Ross R., Kang J., Stout J. R., Wise J. A., (2008a), Short-duration β-alanine supplementation increases training volume and reduces subjective feelings of fatigue in college football players. Nutr Res 28(1):31-35; Hoffman J. R., Ratamess N. A., Kang J., Mangine G., Faigenbaum A. D., Stout J. R. (2006), Effect of creatine and β-alanine supplementation on performance and endocrine responses in strength/power athletes. Int J Sport Nutr Exerc Metabab 16(4):430-446; Hoffman J. R., Ratamess N. A., Ross R., Kang J., Magrelli J., Neese K., Faigenbaum A. D., Wise J. A. (2008b), β-Alanine and the hormonal response to exercise. Int J Sports Med 29(12):952-958; Jordan T., Lukaszuk J., Misic M., Umoren J., Effects of beta-alanine supplementation on the onset of blood lactate accumulation (OBLA) during treadmill running: Pre/post 2 treatment experimental design. J Int Soci Sports Nutr 7: 20-27, 2010; Kendrick I. P., Harris R. C., Kim H. J., Kim C. K., Dang V. H., Lam T. Q., Bui T. T., Smith M., Wise J. A. (2008), The effects of 10 weeks of resistance training combined with beta-alanine supplementation on whole body strength, force production, muscular endurance and body composition. Amino Acids. 34(4):547-554; Kern B. D., Robinson T. L., Effects of β-alanine supplementation on performance and body composition in collegiate wrestlers and football players. J Strength Cond Res. 2011 25:1804-1815; Knapik, J. J., et al. (2004). “Soldier load carriage: historical, physiological, biomechanical, and medical aspects.” Military Medicine 169(1): 45-56; Lieberman H. R., Bathalon G. P., Falco C. M., Kramer F. M., Morgan C. A. 3rd, Niro P., Severe decrements in cognition function and mood induced by sleep loss, heat, dehydration, and undernutrition during simulated combat. Biol Psychiatry. 57:422-429, 2005; Lieberman H. R., Stavinoha T. B., McGraw S. M., White A., Hadden L. S., Marriott B. P., Use of dietary supplements among active-duty US Army soldiers. Am J Clin Nutr. 92:985-995, 2010; Lieberman H. R., Tharion W. J., Shukitt-Hale B., Speckman K. L., Tulley R. Effect of caffeine, sleep loss, and stress on cognitive performance and mood during U.S. Navy SEAL training. Psychopharmacology. 164:250-261, 2002; Murakami T., Furuse M. (2010), The impact of taurine- and beta-alanine-supplemented diets on behavioral and neurochemical parameters in mice: antidepressant versus anxiolytic-like effects. Amino Acids. 39:427-434; Nibbeling, N., et al. (2014). “The effects of anxiety and exercise-induced fatigue on shooting accuracy and cognitive performance in infantry soldiers.” Ergonomics(ahead-of-print): 1-14; Nindl B. C., Barnes B. R., Alemany J. A., Frykman P. N., Shippee R. L., Friedl K. E., Physiological consequences of U.S. army ranger training. Med. Sci. Sports Exerc. 39:1380-1387, 2007; Nindl, B. C., et al. (2013). “Physiological Employment Standards III: physiological challenges and consequences encountered during international military deployments.” European journal of applied physiology 113(11): 2655-2672; Nindl, B. C., et al. (2002). “Physical performance responses during 72 h of military operational stress.” Medicine and science in sports and exercise 34(11): 1814-1822; Russo M. B., Arnett M. V., Thomas M. L., Caldwell J. A., Ethical use of cogniceuticals in the militaries of democratic nations. Amer. J. Bioethics. 8:39-49, 2008; Soher, B. J., et al. (1996). “Quantitative proton MR spectroscopic imaging of the human brain.” Magnetic resonance in medicine 35(3): 356-363; Stellingwerff, T., et al. (2012). “Effect of two β-alanine dosing protocols on muscle carnosine synthesis and washout.” Amino acids 42(6): 2461-2472; Stout J. R., Cramer J. T., Mielke M., O'Kroy J., Torok D. J., Zoeller R. F. (2006), Effects of twenty-eight days of β-alanine and creatine monohydrate supplementation on the physical working capacity at neuromuscular fatigue threshold. J Strength Cond Res 20(4)928-931; Stout J. R., Cramer J. T., Zoeller R. F., Torok D., Costa P., Hoffman J. R., Harris R. C., O'Kroy J. (2007), Effects of β-alanine supplementation on the onset of neuromuscular fatigue and ventilatory threshold in women. Amino Acids. 32(3):381-386; Stout J. R., Graves S. B., Smith A. E., Hartmen M. J., Cramer J. T., Beck T. W., Harris R. C. (2008), The effects of beta-alanine supplementation on neuromuscular fatigue in elderly (55-92 years): a double-blinded randomized study. JISSN. 5(21); Van Thienen R., Van Proeyen K., Vanden Eynde B., Puype J., Lefere T., Hespel P., Beta-alanine, improves sprint performance in endurance cycling. Med Sci Sports Exer 41(4):898-903, 2009; Varley, M. C., Fairweather, I. H., and Aughey, R. J., Validity and reliability of GPS for measuring instantaneous velocity during acceleration, deceleration, and constant motion. J Sports Sci 30: 121-127, 2012; Weeks S. R., McAuliffe C. L., DuRussel D., Pasquina P. F., Physiological and psychological fatigue in extreme conditions: The military example. Phys. Med Rehab. 2:438-441, 2010; Wells A. J., Hoffman J. R., Gonzalez A. M., Stout J. R., Fragala M. S., Mangine G. T., McCormack W. P., Jajtner A. R., Townsend J. R., and Robinson IV, E. H., A Supplement Containing Phosphatidylserine Attenuates Post-Exercise Mood Disturbance and Perception of Fatigue in Humans. Nutrition Research. 33:464-472, 2013; Welsh T. T., Alemany J. A., Montain S. J., Frykman P. N., Young A. J., Nindl B. C., Effects of intensified military field training on jumping performance. Int. J Sports Med. 29:45-52, 2008. American Psychiatric Association (1994) Diagnostic and Statistical Manual of Mental Disorders. Fourth edition. Washington D.C. American Psychiatric Association. American Psychiatric Association (2013) Diagnostic and Statistical Manual of Mental Disorders. Fifth edition. Washington D.C. American Psychiatric Association. Bonfanti L, Peretto P, De Marchis S, Fasolo A (1999) Carnosine-related dipeptides in the mammalian brain. Progress in Neurobiology. 59:333-353. Cobum-Litvak PS, Pothakos K, Tata D A, McCloskey D P, Anderson B J (2003) Chronic administration of corticosterone impairs spatial reference memory before spatial working memory in rats. Neurobiol Learn Mem. 80:11-23. Cohen H, Kozlovsky N, Cramer A, Matar M A, Zohar J (2012a) Fuxe K, Cintra A, Agnati L F, Harfstrand A, Wikstrom A C, Okret S, Zoli M, Miller L S, Greene J L, Gustafsson J A (1987). Studies on the cellular localization and distribution of glucocorticoid receptor and estrogen receptor immunoreactivity in the central nervous system of the rat and their relationship to the monoaminergic and peptidergic neurons of the brain. Journal of Steroid Biochemistry. 27:159-170. Kim H, Yi J H, Choi K, Hong S, Shin K S, Kang S J (2014) Regional differences in acute corticosterone-induced dendritic remodeling in the rat brain and their behavioral consequences. BMC Neuroscience. 15:65. Kohen R, Yamamoto Y, Cundy K C, Ames B N (1988) Antioxidant activity of carnosine, homocarnosine, and anserine present in muscle and brain. Proceedings of the National Academy of Sciences. 85:3175-3179. Murakami T, Furuse M (2010) The impact of taurine- and beta-alanine-supplemented diets on behavioral and neurochemical parameters in mice: antidepressant versus anxiolytic-like effects. Amino Acids. 39:427-434. Myers B, McKlveen J M, Herman J P (2014) Glucocorticoid actions on synapses, circuits, and behavior: Implications for the energetics of stress. Frontiers in Neuroendocrinology. 35:180-196. Richardson M P, Strange B A, Dolan R J (2004) Encoding of emotional memories depends on amygdala and hippocampus and their interactions. Nature Neuroscience. 7:278-285. Romeo R D, Waters E M, McEwen B S (2004) Steroid-induced hippocampal synaptic plasticity; sex differences and similarities. Neuron Glia Biology. 1:219-29. Sebastian V, Estil J B, Chen D, Schrott L M, Serrano P A (2013) Acute physiological stress promotes clustering of synaptic markers and alters spine morphology in the hippocampus. Plos One. 8:e79077. Sousa N, Madeira M D, Paula-Barbosa M M (1998)

Effects of corticosterone treatment and rehabilitation on the hippocampal formation of neonatal and adult rats. An unbiased stereological study. Brain Research. 794:199-210. Stewart M G, Davies H A, Sandi C, Kraev I V, Rogachevsky V V, Peddie C J, Rodriguez J J, Cordero M I, Donohue H S, Gabbott P L, Popov V I (2005) Stress suppresses and learning induces plasticity in CA3 of rat hippocampus: a three dimensional ultrastructural study of thrny excrescences and their postsynaptic densities. Neuroscience. 131:43-54. Takeuchi K, Toyohama H, Sakaquchi M (2000) A hyperosmotic stress-induced mRNA of carp cell encodes NA(+)- and Cl(−)-dependent high affinity taurine transporter. Biochiica et Biophysica Acta. 1464:219-230. Tomonaga S, Kaji Y, Tachibana T, Denbow D M, Furuse M (2005) Oral administration of β-alanine modifies carnosine concentrations in the muscles and brains of chicken. Animal Science Journal. 76:249-254. Tomonaga S, Matsumoto M, Furuse M (2012) β-alanine enhances brain and muscle carnosine levels in broiler chicks. The Journal of Poultry Science. 49:308-312. Tomonaga S, Tachibana T, Takagi T, Saito ES, Zhang R, Denbow D M, Furuse M (2004) Effect of central administration of carnosine and its constituents on behaviors in chicks. Brain Research Bulletin. 63:75-82. Tomonaga S, Yamane H, Onitsuka E, Yamada S, Sato M, Takahata Y, Morimatsu F, Furuse M (2008) Carnosine-induced anti-depressant-like activity in rats. Pharmacology, Biochemistry and Behavior. 89:627-632. Yao S Y, Lau B W, Tong J B, Wong R, Ching Y P, Qui G, Tang S W, Lee T M C, So K F (2011) Hippocampal neurogenesis and dendritic plasticity support running-improved spatial learning and depression-like behavior in stressed rats. PloS One. 2011; 6:e24263.

### DETAILED DESCRIPTION OF THE PREFERRED EMBODIMENTS

Methods and embodiments are described for dietary supplementation with the free amino acid beta-alanine, or salt or ester thereof, and are for illustrative purposes only. Free amino acid beta-alanine, or a salt or ester thereof, may be administered to an individual as a dietary supplement over a period of time in an amount effective to improve physiological conditions related to pregnancy and/or improve physiological conditions of offspring of the individual. The methods described herein may be used for many different industries, including, for example, healthcare, agriculture and many others. The following provides further description of certain embodiments of the invention. As described and claimed here, certain terms are defined and used interchangeably. In the claims set forth, however, references to beta-alanine are meant to embrace the free amino acid beta alanine or a salt thereof. In the claims, when beta alanine is intended to include esters of beta alanine this will be specifically called-out.

As used herein, “β-alanine”, “beta-alanine”, and “BA” are meant to represent the amino acid beta-alanine that is a free amino acid, or a salt or ester of the free amino acid. As will be understood, these terms are to be used interchangeably except as otherwise specified herein. Unless specified otherwise herein, the use of these interchangeable terms does not encompass beta-alanine as a component of a dipeptide, oligopeptide, or polypeptide. Consequently, a dietary supplement containing a dipeptide, oligopeptide, or polypeptide without any free amino acid beta-alanine, or an ester or salt thereof, would not be within the scope of the present invention. For example, a dietary supplement of carnosine, or the like, without any free amino acid beta-alanine, would not be within the scope of the present invention. If, however, a dietary supplement comprises a dipeptide, oligopeptide, or polypeptide in combination with the free amino acid beta-alanine, or an ester or salt thereof, then such dietary supplement would be within the scope of the present invention, provided the free amino acid beta-alanine, or an ester or salt thereof, is present in an effective amount as defined herein. Naturally, the ester forms of the free amino acid beta-alanine, and their salts, could be used in a similar manner, although those forms are not in these originally submitted claims. Additionally, the use of these interchangeable terms in describing the dietary supplement of the invention does not encompass beta-alanine from a natural or conventional food or food product unless otherwise specifically stated or claimed. Natural or conventional foods or food products include, but are not limited to, beef, pork, chicken, meat extract supplements, and predigested meat/protein supplements, and the various essences of meats. Under these definitions, the term “dietary supplement” does not encompass, and does not mean, a natural or conventional food or food product, such as chicken meat, meat essences, chicken broth or meat flavoring.

Furthermore, dietary supplements of the present invention do not encompass pharmaceutical compositions, and the methods of the present invention do not encompass therapeutic treatments, unless specifically set forth in the claims as a pharmaceutical composition. The dietary supplements described herein are non-pharmaceutical compositions and methods for improving physiological conditions related to pregnancy and development of offspring. While the embodiments described herein may utilize pharmaceutical grade ingredients for human consumption and other uses, the dietary supplements and associated approaches are non-pharmaceutical. The embodiments described herein are intended for use as dietary supplements only.

As used herein, the term “human dietary supplement” is intended to mean a dietary supplement as defined under the Dietary Supplement Health and Education Act of 1994 (“DSHEA”). A human dietary supplement as used herein, also means a dietary supplement that is administered or taken by an individual more than once with the purpose of supplementing the diet to increase and/or maintain a component (e.g., beta-alanine) of the supplement, or a substance comprising a component of the supplement (e.g., carnosine) in the body at a higher level(s) than that naturally occurring through natural or conventional meals. Additionally, human dietary supplement further means an addition to the human diet in a pill, capsule, tablet, powder, or liquid form, which is not part of a natural or conventional food or food product, and which effectively increases the function of tissues when consumed.

Dietary supplements for non-humans also means a dietary supplement that is administered or taken by an individual more than once with the purpose of supplementing the diet to increase and/or maintain a component (e.g., beta-alanine) of the supplement, or a substance comprising a component of the supplement (e.g., carnosine) in the body at a higher level(s) than that naturally occurring through natural or conventional meals. Additionally, dietary supplement further means an addition to the diet in a pill, capsule, tablet, powder, or liquid form, which is not part of a natural or conventional food or food product, and which effectively increases the function of tissues when consumed. Dietary supplement may mean a feed supplement whereby a quantity of beta-alanine is included in or added to feed given, typically as a quantity of beta-alanine per amount of feed. In certain embodiments, the supplemented feed may have a predetermined amount of beta-alanine per unit of feed (w/w, w/v, v/v, etc.). The predetermined amount of beta-alanine may provide a desired dose of beta-alanine per feeding. For example, certain livestock may be given a predetermined and/or approximate quantity of feed one or more times per day, week, month, etc. Beta-alanine may be added to the feed to create a supplemented feed and/or fortified feed such that if the livestock consumes the provided feed the livestock will also consume the desired amount of beta-alanine. The amount of supplemented feed for livestock, and thus the amount of beta-alanine provided, may be determined based on the size and/or weight of the livestock. For purposes of this disclosure, a dose, a dosing, etc. may mean a quantity of supplemented feed provided to livestock.

For purposes of this disclosure, “non-human” is intended to mean any living organism that is not a human. This includes, but is not limited to, livestock, pets, zoo animals, etc. Livestock may include, but is not limited to, pigs, fowl, poultry, other birds, cattle, horses, camelids, dogs, cats, etc. Poultry may exclude turkey in certain embodiments. In certain embodiments, “non-human” may exclude horses.

As used herein, the term “period of time”, “over time” or “duration of time” means more than a single dosing, taking or administration of the dietary supplement over an effective time period. The effective time period may be a time during which more than a single dosing has the desired effect on the user. For example, taking a single dose in one day and not taking another dose again does not fall within the definition of “period of time”, “over time” or “duration of time”. Similarly, taking a single dose in one day and not taking another dose again for three months would not fall within the definition of “period of time”, “over time” or “duration of time” as this widely spaced dosing would not create the desired effect on the user. Certain embodiments may not cover instances of a one-time, single dosing that is ineffective at increasing and/or maintaining levels of free amino acid beta-alanine or the resulting levels of carnosine above the normal levels obtained through natural feeding and consumption. More specifically, these terms mean the dietary supplement is taken one or more times per day over a period of seven or more days, wherein generally no two consecutive days pass without the dietary supplementation and the individual supplements the diet at least 3 or 4 days in any 7 day period, more preferably 4 or more days in any 7 day period, more preferably 5 or more days in any 7 day period, more preferably 6 or more days in any 7 day period, more preferably 7 consecutive days in any 7 day period. For example, the individual can take the dietary supplement every day, wherein the dietary supplement is provided incrementally over the course of the day or the individual may take a single dose of the dietary supplement for each day. The individual may also account for non-supplementation days as described above regarding days without supplementation. The period of time described herein can be continued for at least 7 days to about 240 days; preferably about 14 days to about 210 days; more preferably about 21 days to about 180 days; more preferably about 28 days to about 180 days; more preferably about 28 days to about 60 days; even more preferably about 30 days. The period of time described herein can also be that of the gestational period of the non-human animal receiving the dietary supplement as described herein. The period of time may begin before, at, near, or at any time during the gestational period of the non-human animal receiving the dietary supplement as described herein. It will be understood by those of skill in the art, that the period of time can be adjusted by the individual depending on the desired level of results to be achieved and/or maintained.

As used herein, the term “effective amount” or “amount effective to” refers to an amount of the supplement required to achieve the increases or improvements sought and is an amount that is more than contained in the average diet for each species receiving the dietary supplement. For example, omnivores consume about 50-300 mg of carnosine per day and the cooking procedures used would lead to a beta-alanine amount lower than this. It will be understood by those skilled in the art that a one time, single dosing of beta-alanine is incapable of achieving an effective amount for the purposes of dietary supplementation with beta-alanine. Furthermore, it will be understood by those skilled in the art that administering a single dose followed with multiple consecutive days of non-dosing or non-supplementation will not achieve the effective amount as described in the invention. It is also understood that livestock do not generally consume carnosine because carnosine is typically found in meat and livestock to not generally consumer meat.

As used herein, the terms “pregnancy”, “pregnant”, etc. refer to, but are not limited to, a physiological condition of carrying a developing embryo or fetus within the female body.

As used herein, the term “development”, “developing”, etc. refer to, but are not limited to, a changing physiological condition of fetus prior to and/or the offspring after birth. The changing physiological conditions of the fetus and/or offspring may include, but are not limited to, increasing the size of a litter, reducing the number of non-live births, reducing infant mortality, increasing early survival rates, increasing hatchling viability, increasing intelligence, promoting early growth, increasing lean muscle, enhanced nutritive value of offspring, etc. Changing physiological conditions may be measured relative to a standard or threshold. For example, an increased litter size may be determined relative to an average litter size for similar livestock without beta-alanine supplementation. Increased litter size may be an average litter increase of approximately 5%, 10%, 15%, etc. compared to similar livestock without beta-alanine supplementation.

The invention provides an important understanding of how a nutrient or dietary supplement provided over a period of time can improve physiological conditions related to pregnancy and/or development of offspring.

### Forms and Formulations

Administration of the beta-alanine can be as the free amino acid beta-alanine, wherein the free amino acid is not part of a dipeptide, oligopeptide or polypeptide. The free amino acid can be an ester or salt of beta-alanine. The free amino acid can be in a pill, tablet, capsule, granule or powder form. The free amino acid can be administered as part of a solid, liquid or semi-liquid. The free amino acid can be administered as part of a drink (e.g., sports drink) or a food (e.g., health bar). The free amino acid can be administered as part of a supplemented or fortified feed.

The beta-alanine may also be administered in a sustained release formulation, wherein the free amino acid beta-alanine is not part of a dipeptide, oligopeptide, or polypeptide. The beta-alanine administered in a sustained release formulation may also be present as an ester or salt of the beta-alanine. The sustained release formulation can be in a tablet, capsule, granule or powder form. The sustained release formulation can be administered as part of a solid, liquid or semi-liquid. The sustained release formulation of the free amino acid beta-alanine can be administered as part of a drink (e.g., sports drink) or a food or food matrix (e.g., health or energy bar or energy gel). It has been reported that some individuals may experience a slight flushing/tingling of the skin when taking β-alanine as a free amino acid. While this sensation may be uncomfortable, it typically lasts less than 60 minutes. The use of sustained-release forms of beta-alanine has been shown to inhibit, decrease and/or eliminate the flushing/tingling of the skin.

In various embodiments of the present invention, the dietary supplement may be administered (e.g., consumed or ingested) in combination with other ingredients. For example, the free amino acid beta-alanine, or an ester or salt thereof, may be administered in combination with creatine, wherein the creatine is in the form of creatine-monohydrate or other acceptable forms of creatine. Creatine is desirable due to the enhanced ergogenic effect of the formulations of the current invention.

In another embodiment, the dietary supplement comprising the free beta-alanine can further comprise one or more carbohydrates, including simple carbohydrates, for example. Additionally, carbohydrates can include starch and/or sugars, e.g., glucose, fructose, galactose, sucrose, and maltose. The sugars or other carbohydrates can be from various forms of honey, molasses, syrup (e.g., corn syrup, glucose syrup), treacle or gels. It will be understood that the dietary supplement of the invention may comprise one or more carbohydrates in combination with the other ingredients disclosed herein and as part of the forms and formulations defined by the present invention.

In addition, the dietary supplements of the present invention may further comprise insulin, insulin mimics, and/or insulin-action modifiers. Insulin mimics include, but are not limited to, D-pinitol (3-O-methyl-chiroinositol), 4-hydroxy isoleucine, L783,281 (a demethyl-asterriquinone B-1 compound), alpha lipoic acid, R-alpha lipoic acid, guanidiniopropionic acid, vanadium compounds such as vanadyl sulfate or vanadium complexes such as peroxovanadium, and synthetic phosphoinositolglycans (PIG peptides). Insulin-action modifiers that enhance or inhibit the action of insulin in the body, can include, but are not limited to, sulphonylureas, thiazolidinediones, and biguanides. Additionally, the dietary supplements may comprise insulin stimulating agents (e.g., glucose).

In another embodiment, the dietary supplement comprising the free beta-alanine can further comprise one or more electrolytes and/or vitamins (e.g., vitamins B6, B12, E, C, and thiamin, riboflavin, niacin, folic acid, biotin and pantothenic acid). In other embodiments, the dietary supplement may comprise lipids, other amino acids, fiber, trace elements colorings, flavors, natural and/or artificial sweeteners, natural health improving substances, anti-oxidants, stabilizers, preservatives, and buffers.

In certain other embodiments, the dietary supplement of the present invention may comprise other ingredients, for example, anti-oxidants, alpha-lipoic acid, tocotrienols, N-acetylcysteine, co-enzyme Q-10, extracts of rosemary such as carnosol, botanical anti-oxidants such as green tea polyphenols, grape seed extract, COX-1 type inhibitors such as resveratrol, ginkgo biloba, pterostilbene and garlic extracts. Other amino acids such as L-histidine, L-cysteine and/or L-citrulline may be added. In some embodiments, the present invention may comprise combination with an acetylcholine precursor such as choline chloride or phosphatidylcholine may be desirable, for example, to enhance vasodilation. The invention also provides for dietary supplements comprising the free amino acid beta-alanine in combination with such other ingredients as minerals and trace elements in any type or form suitable for human or non-human consumption. It is convenient to provide calcium and potassium in the form of their gluconates, phosphates or hydrogen phosphates, and magnesium as the oxide or carbonate, chromium as chromium picolinate, selenium as sodium selenite or selenate, and zinc as zinc gluconate.

The ingredients, compounds and components disclosed herein as optionally being in the dietary supplement comprising the free amino acid beta-alanine, may be in any combination as part of the dietary supplement. This will be readily understood by those of skill in the field of dietary supplementation.

Once the levels of beta-alanylhistidine have been increased by use of effective amounts of the dietary supplement, otherwise known as a loading phase, the dosing can be adjusted to maintain the levels of beta-alanylhistidine necessary to improve physiological conditions related to pregnancy and/or development of offspring for the purposes of this invention.

The forms and formulations, provided herein, can be those as described and provided for in U.S. Pat. Nos. 5,965,596, 6,426,361, 7,825,084, 8,067,381, and 8,329,207, each of which is incorporated by reference in its entirety.

In one aspect, the dietary supplement is formulated for one or more servings that can be ingested one or more times per day to achieve an effective amount as required by the present invention. Thus, the total daily intake amount required to meet an effective amount of free beta-alanine, or an ester or salt thereof, can be obtained through a single serving or through multiple smaller servings throughout the day that in total meet the required amount of free beta-alanine, or an ester or salt thereof, to be an effective amount in a total daily intake of the dietary supplement. Therefore, a dietary supplement can be formulated with lower amounts of free beta-alanine, or an ester or salt thereof, for the purpose of multiple servings in a day, wherein the total amount through multiple servings meets the desired total daily intake to be an effective amount as defined by the present invention.

The beta-alanine may be administered at a concentration of approximately 100 g/kg of composition. In certain embodiments, the concentration may be approximately 70 g/kg—approximately 130 g/kg, approximately 80 g/kg—approximately 120 g/kg, and/or approximately 90 g/kg—approximately 110 g/kg.

The beta-alanine may be provided as a mixture (composition) of free amino acid beta-alanine, or an ester or salt thereof, and one or more other compounds. The one or more other compounds may be, but is not limited to, glucomannan. In certain embodiments, the mixture may be approximately 80% free beta-alanine, or an ester or salt thereof, and approximately 20% glucomannan. In certain embodiments, the percentage of free beta-alanine, or an ester or salt thereof, may be from approximately 50%—approximately 95%, approximately 60%—approximately 90%, and/or approximately 70%—approximately 85%.

The mixture may be provided as a solution. The solution may include approximately 1.0-1.2 grams of composition per approximately 100 ml water.

The total daily intake amount of the free amino acid beta-alanine, or an ester or salt thereof, is in a range of about 10 mg/kg to about 500 mg/kg; preferably about 50 mg/kg to about 400 mg/kg; more preferably about 75 mg/kg to about 200 mg/kg; and even more preferably about 90 mg/kg to about 110 mg/kg. As described in the present invention, the total daily intake amount in these ranges can be achieved through a single serving formulation comprising the desired effective amount of free beta-alanine. Alternatively, the total daily intake amount in these ranges can be achieved through a formulation for multiple servings, each comprising an amount of the free beta-alanine that when totaled for the day will be within the desired range for a total daily intake delivering an effective amount as defined by the present invention.

Where the dietary supplement is formulated for multiple servings per day within the ranges described herein, it will be understood that there can be 2-12 servings or more, depending on the amounts of free beta-alanine, or ester or salt thereof, in the formulated units. For example, a sustained release tablet comprising 2.0 g of free beta-alanine can be served 3 times per day for a total daily intake of 6.0 g of free beta-alanine. As another example, a formulation comprising 0.5 g of free beta-alanine can be taken 12 times throughout the day for a total daily intake of 6.0 g. This aspect of the present invention applies whether 12 tablets comprising 0.5 g of free beta-alanine are taken at 12 different times throughout the day or if 4 tablets are taken at 3 different times throughout the day. As will be understood in the present invention, it is the total daily intake of the free beta-alanine that must be an effective amount as defined by the present invention. Moreover, the effective amounts in the ranges provided herein account for non-supplementation days as defined by the present invention. Therefore, as long as the individual supplements his/her diet as described herein, the total daily intake of the dietary supplement accounts for non-supplementation days and achieves an effective amount as required over time.

These ranges for total daily intake of free beta-alanine can also account for the various body sizes. Therefore, it will be understood that individuals with smaller body types can take less or more depending on the desired performance levels to be achieved. Likewise, the ranges for total daily intake account for individuals with larger body types that might require a higher total daily intake to achieve the desired performance levels. Regardless of body type, the total daily intake of the effective amounts of free beta-alanine, or an ester or salt thereof, in the dietary supplements of the present invention can account for adjustments in amounts based on the individual's body type requirements and desired performance levels.

It will also be understood that an effective amount being consumed, as defined herein, can be adjusted up or down as long as the total daily intake of free beta-alanine, or an ester or salt thereof, is maintained within the ranges provided herein and meet the definition of effective amounts of the present invention. For example, an individual taking a dietary supplement of the present invention in a formulation delivering a total daily intake of 10 g or 20 g, can adjust the level of supplementation down to 5 g, 6 g, 8 g or 9 g of a total daily intake of the free amino acid beta-alanine, or an ester or salt thereof This is referred to as a maintenance phase. It will be understood by those of skill in the art that individuals may reach a desired level of performance through the dietary supplementation of the present invention and then the individual can opt to reduce the effective amount to a lower effective amount of the present invention to maintain the level of performance achieved. In a converse example, an individual taking a total daily intake of 10 g or 20 g of the free beta-alanine, or an ester or salt thereof, as an effective amount, can increase the total daily intake of the free beta-alanine to any effective amount within the ranges described herein. For example, the individual could increase the total daily intake from 5 g, 6 g, 8 g or 9 g to a total daily intake of 10 g or 20 g, if a further increase in performance is desired. These examples of adjusting the total daily intake of the free beta-alanine described herein are intended as examples of how an individual can increase the level of performance or maintain an achieved level of performance, and these examples are not intended to be limiting on the present invention.

It will also be understood from the present disclosure that an individual can cycle the intake of an effective amount of the free beta-alanine between higher and lower total daily intakes of an effective amount of beta-alanine. For example, an individual could take a total daily amount of 10 g of the free beta-alanine, or an ester or salt thereof, as an effective amount for a period of 28 days, including non-supplementation days, followed by 28 days of taking 5 g of the free beta-alanine, or an ester or salt thereof, as an effective amount, including non-supplementation days, followed by 28 days of taking 10 g of the free beta-alanine, or an ester or salt thereof, as an effective amount, including non-supplementation days. It will also be understood that the time periods and total daily intake amounts given in the example of cycling can be adjusted based on the individual's body type requirements and desired performance levels.

Beta-alanine supplementation may be provided during pregnancy any time after conception.

As will be understood by one of skill in the art through the disclosure of the present invention, other ingredients, e.g., creatine precursors, L-ornithine, glycocyamine, guanidinoacetate, guanidinoacetic acid (GAA), creatine, guanidine, anserine, arginine, glycine, methyl ureido acetic acid, amino acids, and carbohydrates, can be present in the dietary supplement in similar amounts as that described for the free amino acid beta-alanine, or esters or salts thereof.

In certain embodiments, creatine may be administered without beta-alanine for improving physiological conditions related to pregnancy and/or development of offspring. As an example, for improving survivability of offspring creatine may be administered without co-administration of beta-alanine.

The inventors herein have discovered that the use of beta-alanine as a dietary supplement in pregnant individuals may have unexpected benefits. In particular, there may be benefits to administering beta-alanine alone and/or in combination with other compounds, such as creatine, over those benefits achieved by supplementation with carnosine. While not wanting to be constrained by any present theory, it is believed that the beta alanine, especially when provided with creatine, increases the body's endurance and ability to avoid the harmful qualities that can sometimes arise due to low oxygen tension that can be found in pregnancy, childbirth, hatching, and development of offspring. Alleviating some of these problems leads to a greater survival rate of offspring and a protection of their neurological function. Other benefits are also possible.

For both human and non-human individuals, administration of beta-alanine as a dietary supplement may provide unexpected benefits both to fetuses and newborn babies.

In humans, beta-alanine supplementation may provide for improved conditions in offspring. Such improved conditions may include, but are not limited to, improved survival rates, improved rates of live births, improved early infant and/or childhood survival levels, improved physical and/or physiological development, increased intelligence levels in offspring, increased physical abilities in offspring, improved mental functioning, reduction in autism risk and/or severity of autism, improved immune responses, improved cardiac function, improved respiratory function, reduced risk and/or severity of allergies, improved eye-hand coordination, improved overall health, etc.

In non-humans, beta-alanine supplementation may provide for improved conditions in offspring. Such improved conditions may include, but are not limited to, improved survival rates, improved rates of live births, improved early infant and/or childhood survival levels, improved physical and/or physiological development, enhanced nutritive value, enhanced early growth promotion, increased intelligence levels in offspring, increased physical abilities in offspring, improved mental functioning, improved immune responses, improved cardiac function, improved respiratory function, reduced risk and/or severity of allergies, improved overall health, etc. For non-humans raised as a food source, such as pigs and cattle, the improved conditions may also provide for improved lean muscle mass, which may in turn provide for higher quality product, such as leaner bacon. For non-humans, the improved conditions may also include an increased number of live births per litter, a reduction in deaths per litter prior to birth, during birth, and post-birth, an increase in the survival rate of offspring, etc.

In certain embodiments, beta-alanine may be provided as a supplement for a nursing mother. The beta-alanine and/or metabolites of beta-alanine may pass from the mother's milk to the offspring.

Certain embodiments may also include formulation of a supplement feed. Livestock feed may be provided and mixed with beta-alanine. The ratio of feed to beta-alanine may be determined by the weight of the livestock ingesting the supplemented feed and the anticipated quantity of feed to be ingested by the livestock. Certain embodiments may also include formulation of liquids to be ingected by livestock. Beta-alanine may be dissolved and/or suspended in water or other liquids to be ingested by livestock as a supplemented liquid. The ratio of liquid to beta-alanine may be determined by the weight of the livestock ingesting the supplemented liquid and the anticipated quantity of liquid to be ingested by the livestock.

### EXAMPLE 1

Beta-alanine will be used as a dietary supplement for pigs. The study will show an increase in the survivability and an increase in amount of live births per litter for pigs as compared to no supplementation and compared to supplementation with carnosine.

Currently, the number of piglets born per litter in the U.S. at top farms is approximately 10 piglets per litter. The current death rate is approximately 10%. Improvements in number of piglets born per litter and/or reduction in the death rate may have a huge impact in the profitability of farms.

Beta-alanine supplementation will be provided to female pigs during pregnancy. Beta-alanine will be provided in dosages provided in concentrations and/or percentages for times described in this disclosure. Controls will be provided. Controls will include a group of female pigs given no additional supplementation beyond those currently administered during pig pregnancies. Another control group will be provided with supplementation with carnosine at dosages provided in concentrations and/or percentages for times described in this disclosure.

The results will show that the administration of beta-alanine improves the survivability and amount of live births per litter for pigs.

In summary, the results of this study indicate that 30-days or more of beta-alanine supplementation appears to promote improvement in conditions related to pregnancy and development of offspring. The protective effects may be associated with elevations in carnosine levels. Beta-alanine may be used as a dietary supplement for the improvement of conditions related to pregnancy and development of offspring.

### EXAMPLE 2

Beta-alanine will be used as a dietary supplement for pigs and chickens. The study will show an increase in early growth for piglets and chicks as compared to no supplementation and compared to supplementation with carnosine.

Beta-alanine supplementation will be provided to female pigs and female chickens during pregnancy. Beta-alanine will be provided in dosages provided in concentrations and/or percentages for times described in this disclosure. Controls will be provided. Controls will include a group of female pigs and female chickens given no additional supplementation beyond those currently administered during pig and chicken pregnancies. Another control group will be provided with supplementation with carnosine provided in dosages provided in concentrations and/or percentages for times described in this disclosure. The results will show that the administration of beta-alanine improves the early development rate for piglets and chicks.

In summary, the results of this study indicate that 30-days or more of beta-alanine supplementation appears to promote improvement in conditions related to pregnancy and development of offspring. The protective effects may be associated with elevations in carnosine levels. Beta-alanine may be used as a dietary supplement for the improvement of conditions related to pregnancy and development of offspring.

### EXAMPLE 3

Beta-alanine will be used as a dietary supplement for pigs. The study will show an increase in the survivability and an increase in amount of live births per litter for pigs as compared to no supplementation and compared to supplementation with carnosine.

Currently, the number of piglets born per litter in the U.S. at top farms is approximately 10 piglets per litter. The current death rate is approximately 10%. Improvements in number of piglets born per litter and/or reduction in the death rate may have a huge impact in the profitability of farms.

Beta-alanine supplementation will be provided to female pigs prior to and during pregnancy. Beta-alanine will be provided in dosages provided in concentrations and/or percentages for times described in this disclosure. Controls will be provided. Controls will include a group of female pigs given no additional supplementation beyond those currently administered during pig pregnancies. Another control group will be provided with supplementation with carnosine at dosages provided in concentrations and/or percentages for times described in this disclosure.

The results will show that the administration of beta-alanine improves the survivability and amount of live births per litter for pigs.

In summary, the results of this study indicate that 30-days or more of beta-alanine supplementation appears to promote improvement in conditions related to pregnancy and development of offspring. The protective effects may be associated with elevations in carnosine levels. Beta-alanine may be used as a dietary supplement for the improvement of conditions related to pregnancy and development of offspring.

### EXAMPLE 4

Beta-alanine will be used as a dietary supplement for pigs. The study will show an increase in the survivability and an increase in amount of live births per litter for pigs as compared to no supplementation and compared to supplementation with carnosine.

Currently, the number of piglets born per litter in the U.S. at top farms is approximately 10 piglets per litter. The current death rate is approximately 10%. Improvements in number of piglets born per litter and/or reduction in the death rate may have a huge impact in the profitability of farms.

Beta-alanine supplementation will be provided to female pigs starting from breeding age through pregnancy. Beta-alanine will be provided in dosages provided in concentrations and/or percentages for times described in this disclosure. Controls will be provided. Controls will include a group of female pigs given no additional supplementation beyond those currently administered during pig pregnancies. Another control group will be provided with supplementation with carnosine at dosages provided in concentrations and/or percentages for times described in this disclosure.

The results will show that the administration of beta-alanine improves the survivability and amount of live births per litter for pigs.

In summary, the results of this study indicate that 30-days or more of beta-alanine supplementation appears to promote improvement in conditions related to pregnancy and development of offspring. The protective effects may be associated with elevations in carnosine levels. Beta-alanine may be used as a dietary supplement for the improvement of conditions related to pregnancy and development of offspring.

### EXAMPLE 5

Beta-alanine will be used as a dietary supplement for pigs. The study will show an increase in the survivability and an increase in amount of live births per litter for pigs as compared to no supplementation and compared to supplementation with carnosine.

Currently, the number of piglets born per litter in the U.S. at top farms is approximately 10 piglets per litter. The current death rate is approximately 10%. Improvements in number of piglets born per litter and/or reduction in the death rate may have a huge impact in the profitability of farms.

Beta-alanine supplementation will be provided to female pigs from birth through pregnancy. Beta-alanine will be provided in dosages provided in concentrations and/or percentages for times described in this disclosure. Controls will be provided. Controls will include a group of female pigs given no additional supplementation beyond those currently administered during pig pregnancies. Another control group will be provided with supplementation with carnosine at dosages provided in concentrations and/or percentages for times described in this disclosure.

The results will show that the administration of beta-alanine improves the survivability and amount of live births per litter for pigs.

In summary, the results of this study indicate that 30-days or more of beta-alanine supplementation appears to promote improvement in conditions related to pregnancy and development of offspring. The protective effects may be associated with elevations in carnosine levels. Beta-alanine may be used as a dietary supplement for the improvement of conditions related to pregnancy and development of offspring.

Although the foregoing description is directed to the certain embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A process comprising:
administering a dietary supplement comprising a free amino acid beta-alanine, or a salt thereof, over a period of time in an effort to improve one or more physiological responses related to pregnancy.

2. The process of claim 1, wherein the dietary supplement is administered one or more times per day for multiple days.

3. The process of claim 2, wherein the effective amount is at least about 100 mg/kg of bodyweight of the free amino acid beta-alanine, or salt thereof

4. The process of claim 3, wherein the multiple days is at least a fourteen day period.

5. The process of claim 2, wherein the multiple days allows for non-supplementation days.

6. The process of claim 5, wherein the non-supplementation is no more than one consecutive day and no more than two days in a seven day period.

7. The process of claim 1, wherein the dietary supplement is provided with a form of creatine.

8. The process of claim 1, wherein the dietary supplement is provided with an insulin stimulator.

9. The process of claim 1, wherein the dietary supplement is provided with a carbohydrate.

10. The process of claim 1, wherein the dietary supplement is provided with one or more vitamins.

11. The process of claim 1, wherein the dietary supplement is provided with one or more other proteins and amino acids.

12. The process of claim 1, wherein the administering step is during a time when a user is pregnant.

13. The process of claim 1, wherein the dietary supplement is administered to a human.

14. The process of claim 1, wherein the dietary supplement is administered to a non-human.

15. The process of claim 1, wherein the one or more physiological responses are early growth promotion.

16. The process of claim 1, wherein the one or more physiological responses are improved fetal morbidity.

17. The process of claim 1, wherein the one or more physiological responses are improved neonatal morbidity.

18. The process of claim 1, wherein the one or more physiological responses are reduced mortality for offspring.

19. The process of claim 1, wherein the one or more physiological responses are increased early survival rates.

20. The process of claim 1, wherein the one or more physiological responses are improved intelligence in offspring.

21. The process of claim 1, wherein the one or more physiological responses are an increase in the quantity of live births per pregnancy.

21. The process of claim 1, wherein the one or more physiological responses are an increase in lean muscle in offspring.

22. The process of claim 1, wherein the one or more physiological responses are enhanced nutritive value of offspring.

23. A process comprising:
formulating a feed supplement comprising livestock feed and a free amino acid beta-alanine, or a salt thereof, in a effective amount to improve one or more physiological responses related to pregnancy.

24. The process of claim 23, wherein the formulating comprising mixing the livestock feed and the free amino acid beta-alanine, or a salt thereof.

25. The process of claim 23, wherein the feed supplement is administered one or more times per day for multiple days.

26. The process of claim 25, wherein the multiple days is at least a fourteen day period.

27. The process of claim 23, wherein the effective amount is at least about 100 mg/kg of bodyweight of the free amino acid beta-alanine, or salt thereof

28. The process of claim 23, wherein the feed supplement is liquid.

29. The systems and processes described herein.
